(19) United States
(12) Patent Application Publication
(10) Pub. No.: US 2021/0030591 A1
(43) Pub. Date: Feb. 4, 2021

(54) OPHTHALMOLOGICAL SURGICAL SET AND A CONTACT LENS

(71) Applicant: GEUDER AG, Heidelberg (DE)

(72) Inventor: Hamadi EL-AYARI, Frankfurt (DE)

(21) Appl. No.: 16/980,256

(22) PCT Filed: Mar. 6, 2019
(86) PCT No.: PCT/DE2019/200021

§ 371 Date: Sep. 11, 2020

(30) Foreign Application Priority Data
Mar. 12, 2018 (DE) 102018203695.0

(51) Int. Cl. A61F 9/00; A61F 9/007; A61M 1/00

(52) U.S. Cl. CPC A61F 9/0017; A61F 9/007; A61M 2210/0612; A61M 1/0068; A61M 1/008; A61M 1/0039

(57) ABSTRACT

A disclosed ophthalmological surgical set includes a contact lens, a cross-linking substance, and a device configured to generate negative pressure. The contact lens has a deformation side that is configured to face the cornea of an eye when the contact lens is placed on the eye. The deformation side of the contact lens has a shape that is a negative of a substantially emmetropic corneal shape. The cross-linking substance is applied to the cornea before placement of the contact lens on the eye. The contact lens includes a coupling device configured to generate a negative pressure between the contact lens and the eye on which the contact lens is placed such that the cornea can be suctioned, via the negative pressure, onto the deformation side of the contact lens thereby deforming the cornea into the substantially emmetropic corneal shape.

This application is a national stage entry under 35 U.S.C. 371 of PCT Patent Application No. PCT/DE2019/200021, filed Mar. 6, 2019, which claims priority to German Patent Application No. 10 2018 203 695.0, filed Mar. 12, 2018, the entire contents of each of which is incorporated herein by reference.

This disclosure relates to an ophthalmological surgical set. Furthermore, this disclosure relates to a contact lens for use in such an ophthalmological surgical set.

Different methods for correcting defective vision are known in the field. Spectacle lenses and contact lenses represent the simplest option here. However, they are often considered to be extremely irritating and/or uncomfortable and are expensive to obtain and maintain.

Within the scope of refractive surgery, the total refractive power of the optical system of the eye is optimized to the extent that the environment is depicted in focus on the retina such that, in the ideal case, vision aids can be dispensed with. Laser procedures, for example Laser-Assisted In Situ Keratomileusis (LASIK) and Laser Epithelial Keratomileusis (LASEK), represent the majority of refractive interventions. In these procedures, tissue from the cornea is ablated in order to modify the corneal curvature.

All the methods of refractive surgery have the disadvantage that they are invasive interventions. This is associated with corresponding risks, for example, the occurrence of inflammation, a weakness in the cornea, or the FLAP will no longer fuse together. Moreover, such interventions are associated with pain for patients.

Furthermore, it is known in the field to treat defective vision by so-called orthokeratology. In this case, a specially configured shape-stable contact lens is produced for the patient which exerts pressure on the cornea at certain places in advance. The cornea is specifically deformed due to this pressure such that the refraction changes. Corresponding contact lenses are usually only worn at night so that the user can get through the day without a vision aid. A significant disadvantage of this method is that it is a chronologically limited correction of the defective vision, i.e. the process of cornea deformation is reversible. The visual acuity worsens again throughout the day such that, for example, driving a vehicle without a vision aid late in the evening is to be avoided. In addition, the contact lenses normally have to be worn every night. Thus, the object of the disclosure is to provide a correction of the defective vision with a simple system and in a manner that is gentle for patients.

According to the disclosure, said object is achieved by the features of claim 1. Accordingly, an ophthalmological surgical set includes at least one contact lens and at least one cross-linking substance for the cornea, wherein a deformation side, facing the cornea, of the contact lens is embodied as a negative of an at least substantially emmetropic corneal shape and wherein the contact lens includes a device for providing negative pressure between the contact lens and the eye wearing the contact lens such that the cornea can be suctioned onto the deformation side and consequently can be brought into the at least substantially emmetropic corneal shape.

In a manner according to the disclosure, it has initially been determined that a correction of the defective vision can occur in a surprisingly simple manner in that the cornea is placed into an emmetropic shape and retained in this shape by a cross-linking substance. To this end, a contact lens is provided further according to the disclosure, which functions substantially like an orthokeratological contact lens, wherein devices are provided for generating a negative pressure between the deformation side of the contact lens and the cornea. It is thereby possible to suction the cornea onto the deformation side and thus to achieve the desired, at least substantially emmetropic corneal shape in the shortest time. In order to keep or “freeze” the cornea in this state, the cross-linking substance is applied to the cornea in advance. The cross-linking substance in this case does not have to be in pure form; rather, it may be a pharmaceutical composition which also contains one or more other components, in addition to the at least one cross-linking substance. While the cornea is kept in the desired shape by the negative pressure, the cross-linking process brought about by the cross-linking substance takes place; this means the cornea is cross-linked, whereby the biomechanical resistance (similar to the modulus of elasticity) of the cornea must be increased drastically. A connection for a fluid-connection element may be provided such that negative pressure can be created between the deformation side and the cornea via the fluid-connection element, for example a tube.

The surgical set according to the disclosure thus makes it possible to place the cornea into a target state in a short time and to keep and/or to fix it in this emmetropic shape without the contact lens having to be worn over a longer time period or at regular intervals.

The emmetropic corneal shape in this case is understood to be the shape of the cornea individually configured to the patient, in which there is normal vision or in which the correctable vision defects are eliminated, at least substantially. The term “normal vision” here is to be understood in the broadest sense and is not to be equated necessarily with 100% visual acuity. Furthermore, it should be noted that the eye in this case may be a human eye. In addition, the term “operation” should be understood in the broadest sense; it may be an intervention by a physician or an optician whereby defective vision is intended to be eliminated or improved.

With respect to creating the negative pressure with a simple device, the surgical set may include a syringe with a fluid-connection element, for example, a tube connection, wherein the connection of the contact lens to the fluid-connection element can be implemented using a fluid connection. In general, any form of a cylinder-piston mechanism or any other mechanism for providing negative pressure can be connected to the connection via a tube connection.

In an advantageous manner, the syringe, for example, the piston of the syringe, may be pre-tensioned via an elastic element such that a defined negative pressure can be created. The elastic element may be, for example, a spring, for example, a compression spring. For example, the syringe piston may be pre-tensioned such that it is pressed out of the syringe cylinder. The negative pressure can be predetermined via the volume of the syringe cylinder, regarding which reference is made to the thermal equation of state of ideal gases p×V=n×R×T.

In order to implement a link between the connection and a fluid-connection element, for example, a tube, the connection of the contact lens and/or the fluid-connection element may be configured such that a form-fitting and/or force-fitting connection can be implemented. For example, the connection may be implemented as part of a bayonet connection or a Luer-Lock connection. The fluid-connection element may have a connection element corresponding thereto.

With respect to an exact positioning and/or alignment of the contact lens in the eye of the patient, at least one marking may be provided on the contact lens. The marking may be implemented, for example, in the manner of a crosshair or of a reticle of a telescopic sight. In addition, the use of further aids is conceivable, for example mirror projections of the target position via an OP microscope or the use of a slit lamp.

In an especially advantageous manner, the deformation side of the contact lens may be configured to deform the cornea such that at least one zone can be created for correcting farsightedness and at least one zone for correcting nearsightedness.

With respect to the cross-linking substance, it is conceivable that it is a part of a pharmaceutical composition which is suitable for administering onto the eye. According to WO 2017/077300 A1, the composition includes the following:
(i) an aqueous carrier; and
(ii) the non-toxic, water-soluble cross-linking substance, including two or more carboxyl groups and/or one or more anhydride groups and/or one or more ester groups or a pharmaceutically acceptable salt thereof, dissolved in the aqueous carrier; wherein the composition may have a pH in a range of from 6 to 9, provided that the non-toxic, water-soluble cross-linking substance may not be glutaric acid anhydride.

In an advantageous manner, the non-toxic, water-soluble cross-linking substance of the pharmaceutical composition may include 2 to 4 carboxyl groups.

In an advantageous manner, the non-toxic, water-soluble cross-linking substance may be a compound of formula (I) shown below or formula (II) or a pharmaceutically acceptable salt and/or solvate thereof

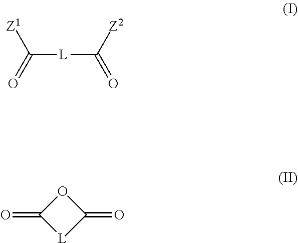

where:
L can be selected from:
   (i) a linear or branched (1-12 C)alkylene linker, which optionally includes one or more heteroatoms, selected from N, O, or S, and is optionally substituted with one or more groups selected from carboxy, anhydride, oxo, halogen, trifluoromethyl, cyano, nitro, hydroxy, mercapto, amino, (1-6 C)alkyl, (1-6 C)alkoxy, (1-6 C)alkylthio, (1-6 C)alkylsulfinyl, (1-6 C)alkylsulfonyl, (1-6 C)alkylamino, di-[(1-6 C)alkyl]-amino, (1-6 C)alkoxycarbonyl, (2-6 C)alkanoyl, or (2-6 C)alkanoyloxy, or
   (ii) a water-soluble polymer chain; and
Z¹ and Z² may be independently selected from OH or, where R may be selected from (1-6 C)alkyl, succinimide, 3-sulfosuccinimide, or pentafluorophenyl.
In an advantageous manner, the non-toxic, water-soluble cross-linking substance may be a compound of formula (I) or formula (II) or a pharmaceutically acceptable salt and/or solvate thereof:

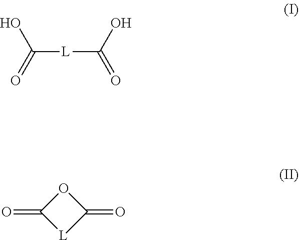

where L
   (i) may be a linear or branched (1-12 C)alkylene linker, which may optionally include one or more heteroatoms, selected from N, O, or S, and may optionally be substituted with one or more groups selected from carboxy, anhydride, oxo, halogen, trifluoromethyl, cyano, nitro, hydroxy, mercapto, amino, (1-6 C)alkyl, (1-6 C)alkoxy, (1-6 C)alkylthio, (1-6 C)alkylsulfinyl, (1-6 C)alkylsulfonyl, (1-6 C)alkylamino, di-[(1-6 C)alkyl]-amino, (1-6 C)alkoxycarbonyl, (2-6 C)alkanoyl, or (2-6 C)alkanoyloxy, or
   (ii) a water-soluble polymer chain.

According to a further advantageous embodiment, it is conceivable that the non-toxic, water-soluble cross-linking substance may be a compound of formula (I) or a pharmaceutically acceptable salt and/or solvate thereof, and where L may be a (2-8 C)alkyl, which is optionally substituted with one or more groups of formula —(CH₂)ₙCO₂H, where n may be an integer between 0 and 10.

In addition, the pharmaceutical composition may be implemented such that the non-toxic, water-soluble cross-linking substance may be selected from sebacic acid, azelaic acid, suberic acid, pimelinic acid, adipic acid, glutaric acid, or succinic acid.

In an advantageous manner, the non-toxic, water-soluble cross-linking substance may be a compound of formula (I) or a pharmaceutically acceptable salt and/or solvate thereof, and L may be a polyethylene glycol chain.

The pharmaceutical composition may additionally be the non-toxic, water-soluble cross-linking substance bis-(succinimidyl)-penta-(ethylene glycol).

With respect to an advantageous embodiment, it is possible that the pharmaceutical composition further includes a buffer in order to keep the pH of the composition in a pH range of from 6 to 9. Specifically, the buffer can keep the composition in a pH range of from 6.5 to 7.5.

Alternatively or additionally, the pharmaceutical composition may further include one or more peptide coupling reagents and/or include one or more carbodiimide peptide coupling reagents. Moreover, it may be advantageous if the pharmaceutical composition includes an activating agent.

In general, it may be advantageous that the non-toxic, water-soluble cross-linking substance of the disclosure are either completely or partially dissolved in an aqueous carrier. The term “aqueous carrier” may be understood as a fluid carrier which predominantly contains water, for example more than approximately 50% water by volume.

Furthermore, it is possible that the aqueous carrier contains further solvents, for example organic solvents which are completely or partially miscible with water. The aqueous carrier may also include additives which may be ionic, organic, or amphiphilic, for example, surfactants, viscosity modifiers, tonicity agents, sterilization agents, and solubility enhancers.

Any suitable buffer can be selected as the buffer, for example from the group including: buffers based on phosphates, acetates, citrate, sulfonic acid, ascorbate, linolenate, carbonate, and bicarbonate.

The cross-linking of biological material (e.g. collagen) by the previously described pharmaceutical compositions is provided by the reaction of carboxyl (or anhydride) groups of the cross-linking substance with amino functionalities on the biological material. In this case, two or more amide bonds are formed such that the biological material is cross-linked covalently.

Details of specific exemplary embodiments of the pharmaceutical composition can be found in WO 2017/077300 A1 as previously mentioned.

To simplify handling, the cross-linking substance and/or the pharmaceutical composition may be a fluid in a metering mechanism in the surgical set. The metering mechanism may advantageously be a syringe, a pipette, or another device which provides drop-wise application of the cross-linking fluid.

In order to administer the pharmaceutical composition, the metering mechanism may have a first chamber with the pharmaceutical composition and a second chamber with one or more peptide coupling reagents and optionally an activator, dissolved in a suitable pharmaceutically acceptable carrier, wherein the metering mechanism is configured such that it mixes at least a portion of the contents of the first and second chamber either before or during application onto the eye.

In an advantageous manner, the surgical set may contain at least one lid barrier and/or at least one receiving device—for example a swab—for removing excess fluids and/or excess cross-linking substance.

The fundamental object is further achieved by the features of dependent claim 14. Thus, a contact lens is indicated for use in an ophthalmological surgical set according to any of claims 1 to 13, with a deformation side, facing the cornea, which is formed as a negative of an at least substantially emmetropic corneal shape and with a device for providing negative pressure between the deformation side and the eye wearing the contact lens such that the cornea can be suctioned onto the deformation side and consequently can be brought into the at least substantially emmetropic corneal shape.

The contact lens may be produced in an injection-molding process or a microinjection-molding process or also by a machining process, for example turning or milling, or even by a 3-D printing process.

Express reference is made to the extent that the contact lens according to the disclosure in accordance with claim 14 may have, in an advantageous manner, one or more of the features of the contact lens, contained in the ophthalmological surgical set, according to any of claims 1 to 13. Furthermore, the contact lens according to claim 14 may have one or more of the features of the previous description as regards claims 1 to 13 as well as the description of the following treatment procedure.

The disclosure described herein further includes a process for treating defective vision, for example, while using an ophthalmological surgical set according to any of claims 1 to 13 and/or a contact lens according to claim 14, including the following stages:
applying a cross-linking substance to the eye to be treated;
placing the contact lens on the eye to be treated and creating a negative pressure such that the cornea is suctioned to the deformation side of the contact lens:
maintaining the negative pressure until the cross-linking is at least substantially completed;
removing the contact lens from the eye to be treated.

Furthermore, it is conceivable that a lid barrier is first used on the patient and optionally the cornea is dabbed dry so as to not dilute the cross-linking substance to be applied. It is also possible for the intervention to take place under anesthesia if the patient desires this or cannot keep the eye still.

Once the contact lens has been removed from the eye, it may be necessary to flush out any remaining cross-linking substance and/or reagents.

If the patient has been using contact lenses previously to correct the defective vision, it may be advantageous that soft contact lenses not be used at least one week before a preliminary examination and that hard contact lenses not be used at least two weeks before so that the examination results are not distorted. The preliminary examination may include for example, tonometry, in which a measurement of the interior pressure of the eye takes place to exclude glaucoma (also known as “green star” in German). Alternatively or additionally, cornea diagnostics can be implemented in order to determine any degeneration in the cornea as well as weak areas, for example, on the edge of the cornea. To this end, the pupils can be dilated using eyedrops. Alternatively or additionally, visual acuity and refraction can be determined, i.e. an objective and subjective determination of the refractive power of the eyes to be treated (diopter number). Alternatively or additionally, a pachymetric measurement can be used, i.e. a measurement of the corneal thickness via ultrasound examination, and/or biometrics are implemented to measure the length of the eyeball via laser technology (IOL Master). According to a further alternative or additional design of the preliminary examination, a topography can be implemented, i.e. a measurement of the eye surface and/or any corneal curvature and determination of the individually different heights and curvature values. Thus, any possible corneal diseases or corneal scarring can be determined. The previous designs of the preliminary examination are the fundamentals for determining the design of the contact lens according to the disclosure which is to be configured individually to the patient.

By the previously described pachymetry, biometrics, and/or the corneal topography, the target topography of the cornea can be determined which should lead to the desired reactive result after treatment. In this case, a quasi topographic vector map of the cornea is created with precision in the micron range—in the “actual” state and in the ideal “target” state (emmetropic state). This “target” states can be defined such that any correctable astigmatism and primarily the target refraction (sharp picture on the cornea with consideration of eye length, etc.) are achieved.

This target topography can be configured as a “negative impression” (similar to a mold) as a basis for the contact lens to be produced specific to the patient, for example, the deformation side. The contact lens may be produced in an injection-molding process or a microinjection-molding process or also by a machining process, for example turning or milling, or even by a 3-D printing process.

In any case, the contact lens configured individually to the patient is capable—due to the inner curvature or design of the deformation side—of ensuring the desired steepening (hypermetropia=farsightedness) or flattening (myopia=nearsightedness) by use of suctioning of the cornea, and thus represents a simple alternative to a LASIK treatment. The correction of astigmatism and any possible multifocal cross-linking are also conceivable, i.e. the creation of zones for farsightedness and nearsightedness on the cornea.

There are then various options for designing and enhancing the teaching of the disclosure in an advantageous manner. To this end, reference is made, on one hand, to the claims referring back to claim 1 and, on the other hand, to the subsequent explanation of exemplary embodiments of the disclosure with reference to the drawings. In conjunction with the explanation of the exemplary embodiments of the disclosure with reference to the drawings, embodiments and enhancements of the teaching are also generally explained. The drawings show the following:

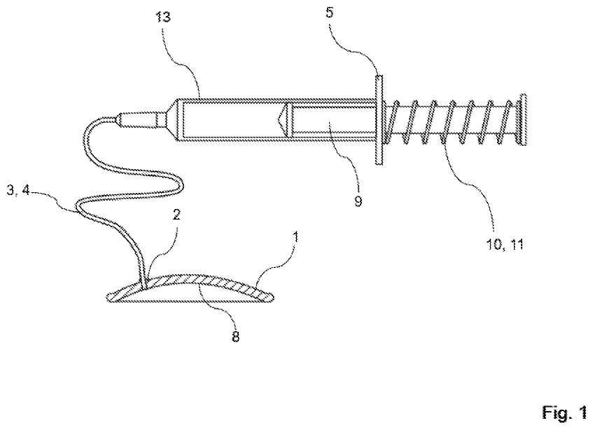

FIG. 1 a schematic, first view of an exemplary embodiment of an ophthalmological surgical set according to the disclosure including a contact lens according to the disclosure;

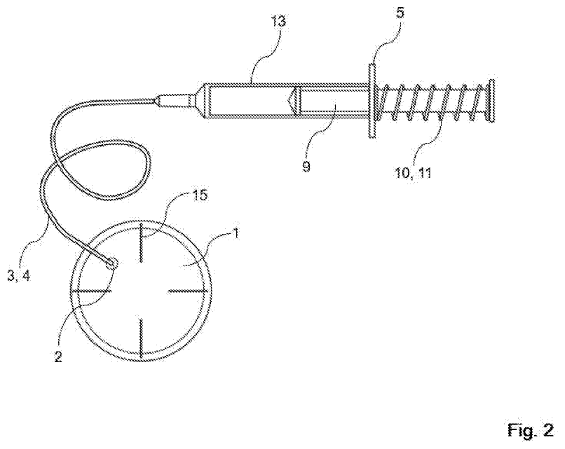

FIG. 2 a schematic, second view of the exemplary embodiment according to FIG. 1;

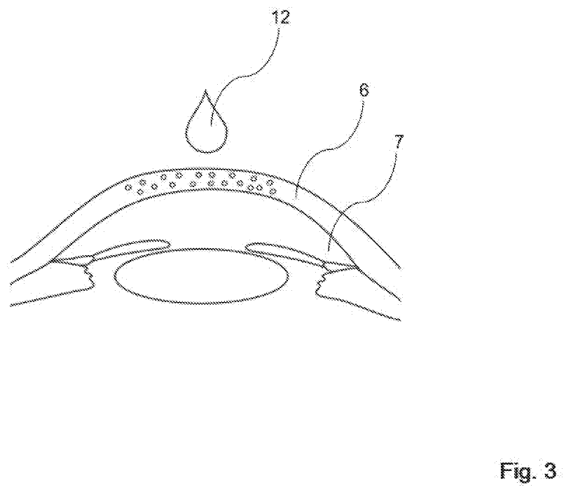

FIG. 3 a schematic view of a section from a human or animal eye, onto which a cross-linking substance of an ophthalmological surgical set according to the disclosure is applied;

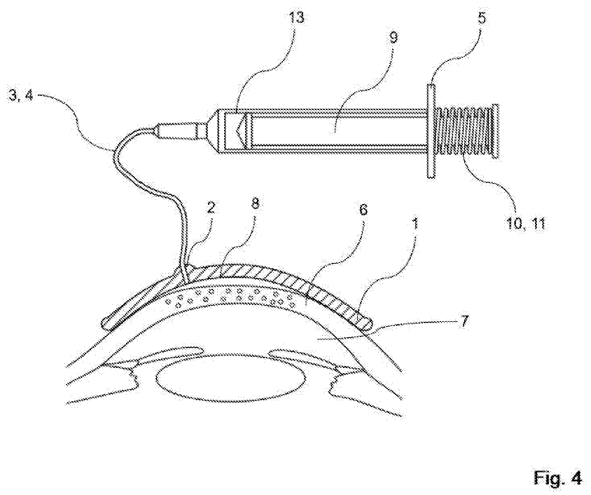

FIG. 4 a further schematic view of a section from a human or animal eye, onto which a contact lens of an ophthalmological surgical set according to the disclosure is applied;

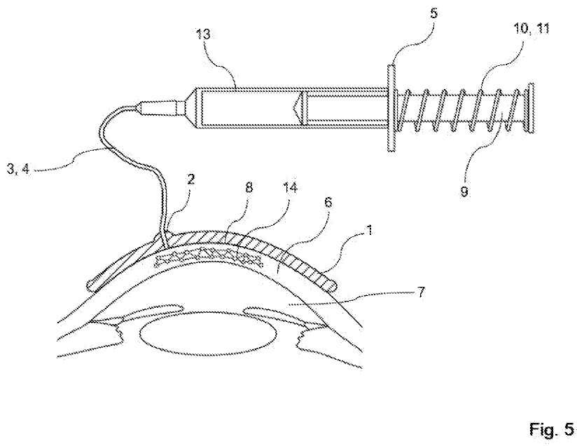

FIG. 5 the view according to FIG. 4, wherein there is a negative pressure between the cornea and the deformation side.

FIGS. 1 and 2 show an exemplary embodiment of a contact lens 1 according to the disclosure of an ophthalmological surgical set according to the disclosure. The contact lens 1 is produced from a shape-stable material and has a device for providing negative pressure configured as a connection 2, to which a tube 4 serving as a fluid-connection element 3 is coupled. The contact lens 1 is connected to a syringe 5 via the tube 4, which syringe is used to create negative pressure between a cornea 6—which is not shown in FIGS. 1 and 2—of the eye 7 to be treated and the deformation side 8 of the contact lens 1.

The deformation side 8 is individually configured to the eye to be treated, that is it corresponds substantially to the negative of an emmetropic corneal shape.

The syringe piston 9 is pre-tensioned via an elastic element 10, i.e. a spring 11. FIG. 2 shows that the contact lens 1 has a marking 15 in the shape of a crosshair or of a reticle. Precise positioning of the contact lens 1 on the eye 7 is thereby made possible for the surgeon in a simple manner.

The use of the surgical set according to the disclosure is explained with reference to FIGS. 3 to 5. First, the cross-linking substance 12 is applied to the cornea 6, for example, as drops. In a next stage, the contact lens 1 individually configured to the eye 7 is inserted, as is shown in FIG. 4. FIG. 4 clearly shows that the syringe piston 9 is pressed against the spring 11 to the extent that at least no substantial negative pressure prevails between the cornea 6 and the deformation side 8.

When the syringe piston 9 is released or let go by the surgeon, the spring 11 presses the syringe piston 9 out of the syringe cylinder 13 such that a defined negative pressure prevails between the deformation side 8 and the cornea 6. This state is shown in FIG. 5, which also shows that the cornea 6 is suctioned onto the deformation side 8 by application of negative pressure. Thus, the cornea 6 is placed and retained in the desired emmetropic corneal shape, in which the cross-linking substance 12 can take effect. The mechanism of action of the cross-linking substance 12 is shown in FIG. 5 by structure 14, which illustrates the cross-linking of the collagen fibers of the eye.

Once the cross-linking substance 12 has taken effect, the negative pressure can be released, and the contact lens 1 can be removed from the eye. By action of the cross-linking substance 12, the biomechanical resistance of the cornea 6 is increased such that the emmetropic corneal shape remains at least for a longer period of time.

With regard to other advantageous embodiments of the surgical set according to the disclosure, to avoid repetition, reference is made to the general part of the description and also to the accompanying claims.

Finally, it is expressly pointed out that the above-described exemplary embodiments of the surgical set according to the disclosure serve only to explain the claimed teaching, but do not restrict it to the exemplary embodiments.

### LIST OF REFERENCE NUMERALS

1 Contact lens
2 Connection
3 Fluid-connection element
4 Tube
5 Syringe
6 Cornea
7 Eye
8 Deformation side
9 Syringe piston
10 Elastic element
11 Spring
12 Cross-linking substance
13 Syringe cylinder
14 Structure
15 Marking

1. An ophthalmological surgical set, comprising:
a contact lens configured to be placed on an eye, the contact lens having a deformation side that is configured to face the cornea of the eye when the contact lens is placed on the eye; and
a cross-linking substance configured to be applied to the cornea before placement of the contact lens on the eye,
wherein the deformation side of the contact lens is configured to have a shape that is a negative of a substantially emmetropic corneal shape, and
wherein the contact lens includes a coupling device configured to generate a negative pressure between the contact lens and the eye on which the contact lens is placed such that the cornea can be suctioned, via the negative pressure, onto the deformation side of the contact lens thereby deforming the cornea into the substantially emmetropic corneal shape.

2. The ophthalmological surgical set according to claim 1, wherein the coupling device is configured to provide a fluidic connection between the contact lens and a fluid-connection element.

3. The ophthalmological surgical set according to claim 2, wherein the coupling device is configured to provide a form-fitting and/or force-fitting connection with the fluid-connection element (3).

4. The ophthalmological surgical set according to claim 2, wherein the coupling device is configured as a bayonet connection or as a Luer-Lock connection.

5. The ophthalmological surgical set according to claim 2, further comprising:
a syringe fluidically coupled to the fluid-connection element,
wherein the syringe is configured to draw fluid from the contact lens, through the coupling device and the fluid-connection element to thereby generate the negative pressure between the deformation side of the contact lens and the cornea.

6. The ophthalmological surgical set according to claim 5, wherein the syringe further comprises an elastic element and a plunger,
wherein the elastic element is pre-tensioned and is in contact with the plunger to thereby generate a force on the plunger of the syringe which generates a pre-determined negative pressure.

7. The ophthalmological surgical set according to claim 1, wherein the contact lens further comprises a marking that is configured as a positioning aid.

8. The ophthalmological surgical set according to claim 1, wherein, the deformation side of the contact lens is configured to have a shape such that at least one zone is generated on the cornea when the cornea is deformed,
wherein the zone is configured to correct farsightedness or nearsightedness.

9. The ophthalmological surgical set according to claim 1, wherein, the cross-linking substance is part of a pharmaceutical composition which is suitable for administration on or onto the eye, the composition comprising:
(i) an aqueous carrier; and
(ii) a non-toxic, water-soluble cross-linking component,
comprising two or more carboxyl groups and/or one or more anhydride groups and/or one or more ester groups or a pharmaceutically acceptable salt thereof, dissolved in the aqueous carrier,
wherein the composition has a pH in a range of from 6 to 9, wherein the non-toxic, water-soluble cross-linking means is not glutaric acid anhydride.

10. The ophthalmological surgical set according to claim 1, wherein the cross-linking substance is provided in a fluid by a metering device.

11. The ophthalmological surgical set according to claim 10, wherein the metering device is a syringe or a pipette.

12. The ophthalmological surgical set according to claim 1, further comprising a lid barrier.

13. The ophthalmological surgical set according to claim 1, further comprising a receiving device configured to remove excess fluids and/or excess cross-linking substance from the eye.

14. A contact lens configured for use in an ophthalmological surgical set, the contact lens comprising:
a deformation side that is configured to face the cornea of an eye when the contact lens is placed on the eye, wherein the deformation side of the contact lens is configured to have a shape that is a negative of a substantially emmetropic corneal shape; and
a coupling device configured to generate a negative pressure between the contact lens and the eye on which the contact lens is placed such that the cornea can be suctioned, via the negative pressure, onto the deformation side of the contact lens thereby deforming the cornea into the substantially emmetropic corneal shape.

15. The ophthalmological surgical set according to claim 2, wherein the fluid-connection element is a tube.

16. The ophthalmological surgical set according to claim 13, wherein the receiving device is a swab.
